(19) United States
(12) Patent Application Publication
(10) Pub. No.: US 2015/0185218 A1
(43) Pub. Date: Jul. 2, 2015

(54) METHODS AND SYSTEM FOR DETECTING MELANOMA

(71) Applicant: Ramot at Tel-Aviv University Ltd., Tel-Aviv (IL)

(72) Inventors: Sefi Vernick, Tel-Aviv (IL); Yosi Shacham-Diamand, Tel-Aviv (IL); Judith Rishpon, Rechovot (IL); Michal Mossberg, Kochav Yair (IL); Ofri Vaisman, Tel-Aviv (IL)

(73) Assignee: Ramot At Tel-Aviv University Ltd., Tel Aviv (IL)

(21) Appl. No.: 14/404,465

(22) PCT Filed: May 30, 2013
(86) PCT No.: PCT/IL13/50467

§ 371 Date: Nov. 27, 2014

Related U.S. Application Data
(60) Provisional application No. 61/653,456, filed on May 31, 2012.
(60) Provisional application No. 61/653,457, filed on May 31, 2012.

(51) Int. Cl. G01N 33/574; G01N 27/327; G01N 33/50

(52) U.S. Cl. CPC G01N 33/5743; G01N 33/5011; G01N 27/327; G01N 2333/90245

(57) ABSTRACT

A method of determining tyrosinase activity of a cell is disclosed. The method comprises:
(a) contacting the cell with a phenol under conditions wherein tyrosinase of the cell catalyzes a reaction with said phenol, so as to generate a product which produces an electrical signal; and
(b) measuring a level of the electrical signal, thereby determining tyrosinase activity of the cell.

Use of the method for detecting melanoma in skin samples is also disclosed.

### FIELD AND BACKGROUND OF THE INVENTION

The present invention, in some embodiments thereof, relates to a method of analyzing tyrosinase activity and, more particularly, but not exclusively, to use of same for detecting melanoma. Some embodiments of the present invention relates to a system for detecting analytes.

Melanoma is the most dangerous type of skin cancer. It is the leading cause of death from skin diseases. Cutaneous malignant melanoma (CMM), which accounted for 62,500 new cases of cancer in 2008, is the sixth most common malignancy in men and the seventh most common in women in the United States. Estimated new cases and deaths from melanoma in the United States in 2012: New cases: 76,250, Deaths: 9,180. Although 80% of new lesions are localized to the skin where effective surgical resections result in more than 95% 5-year survival, disease can recur in individuals with localized lesions despite appropriate management. Because adjuvant therapy is not broadly indicated for localized melanoma due to unfavorable risk-benefit ratios, there is a critical need to identify, at the time of diagnosis, the subset of patients most likely to benefit from adjuvant treatment to improve overall survival outcomes. Although, in addition to localization, nine clinicopathologic prognostic markers have been identified for CMM and have been used to establish clinically validated risk stratifications among melanoma patients (Balch, Soong et al. 2001; Gimotty, Elder et al. 2007), risk models based on these markers do not account for all of the observed variability in melanoma-related survival. Indeed, in melanoma (Bittner, Meitzer et al. 2000; Onken, Ehlers et al. 2006; Winnepenninckx, Lazar et al. 2006) as in other cancers (Golub, Slonim et al. 1999), tumors with identical clinical and histological parameters have markedly different mRNA expression profiles, and tumor subgroups classified by gene expression can be strongly associated with differential survival.

Primary and metastatic melanomas display a variety of morphologic and architectural patterns that mimic other soft tissue tumors and give rise to problems in the differential diagnosis. Adding to the diagnostic difficulties is the focal presence of melanin, which has been described in neurofibromas, schwannomas, DFSPs, and MPNSTs (Boyle, Haupt et al. 2002).

Tyrosinase is a cytoplasmic melanocyte differentiation protein and is a key enzyme in melanin synthesis. Tyrosinase catalyzes the two initial steps in the biosynthetic pathway—i.e., hydroxylation of tyrosine to dopa and oxidation from dopa to dopaquinone. Dopaquinone then enters two separate pathways, leading to the synthesis of eumelanin or pheomelanin (Chen, Stockert et al. 1995). Immunohistochemical studies have consistently shown strong positive staining in tissues of malignant melanomas and varied staining in benign nevi (Jungbluth, Iversen et al. 2000). Tyrosinase has a high sensitivity for melanoma, with early reports of over 85% positivity (Orchard 2000). As previously mentioned, the differential diagnosis of melanoma poses many problems to dermapathologists and the sensitivity of clinical diagnosis of experienced dermatologists was reported to be ˜70% (Garbe, Penis et al. 2010). Moreover, the histological evaluation of melanocytic lesions is subject to considerable variation in opinion between expert dermatopathologists. In a recent study, investigators evaluated the concordance of opinions of two dermatopathologists in an academic setting consulted for difficult cases of melanocytic neoplasms. In such situations, directly impacting patient management, complete agreement was reached only in 54.5% of cases, whereas in 25% of histopathological samples the two diagnoses were highly discordant (Lodha, Saggar et al. 2008). The unaided “naked eye” clinical recognition depends on the clinician's type of training and level of experience, but up to 1.3 and 3.8% of melanomas can be mistaken for benign lesions by dermatologists and non-dermatologists, respectively (Reeck, Chuang et al. 1999). Even within a group of dermatologists in a dedicated pigmentary lesion clinic, the accuracy of correctly identifying melanoma was found to be higher for professionals with more than 10 years of experience than those with 3-5 years and 1-2 years of practice (accuracy of 80, 62, and 56%, respectively) (Alexandrescu, Kauffman et al. 2010).

Electrochemical biosensors for melanoma biomarker detection, carries huge potential for onsite melanoma diagnosis. An effective and low cost diagnostic tool for the detection of functional biomarkers may provide reliable diagnosis of melanoma, substantially improving sensitivity and specificity.

The electrochemical detection of cancer biomarkers via amperometric biosensors is generally carried out by placing cultured cancer cells onto the electrochemical system. Alternatively, the levels of markers secreted into or extracted by the culture medium were detected. In the case of melanoma, however, biomarkers are sometimes displayed within the tumor tissue and are not secreted, requiring the use of biopsy as inoculum for the growth of cell culture, involving a labor intensive, costly, and time consuming procedure.

Direct detection of biopsy samples as is, is therefore highly envisaged. In spite of its potential diagnostic value, this form of bioelectrochemical measurement was not reported.

U.S. Pat. App. No. 20060100488 teaches detection of cancerous cells by directly monitoring the electrical response of the cells following application of an alternating current. WO 91/15595 teaches analysis of electrical conductivity of cancer cells for monitoring responsiveness to therapy and drug screening. Specifically, WO 91/15595 teaches monitoring the effectiveness of a particular agent to inhibit increases in the volume and number of cancer cells by analyzing electrical conductivity thereof. Accordingly, both these patent applications teach that the intrinsic electrical properties of a cancer cell may be used as markers for detection and monitoring of cancer cells.

U.S. Pat. Appl. No. 20040053425 teaches amperometric analysis of an analyte in a fluid, wherein the electrode comprises the current producing enzyme. U.S. Pat. Appl No. 20040053425 does not teach amperometric detection of intracellular markers.

U.S. Pat. No. 5,149,629, teaches amperometric analysis of markers, including cancer cell markers, wherein the electrode comprises antibodies capable of binding the markers thereto. The analysis is by substrate competition. U.S. Pat. No. 5,149,629 does not detect endogenous amperometric features of cancer cells.

U.S. Patent Application No. 20090232740 teaches amperometric detection for diagnosing cancer in cancer cell samples and biopsy samples by analyzing cellular enzymatic activities.

### SUMMARY OF THE INVENTION

According to an aspect of some embodiments of the present invention there is provided a method of determining tyrosinase activity of a cell. The method comprises: (a) contacting the cell with a phenol under conditions wherein tyrosinase of the cell catalyzes a reaction with the phenol, so as to generate a product which produces an electrical signal; and (b) measuring a level of the electrical signal, thereby determining tyrosinase activity of the cell.

According to some embodiments of the invention the method wherein the cell is a skin cell.

According to some embodiments of the invention the cell is a mammalian cell.

According to some embodiments of the invention the phenol comprises tyrosine or 3,4-dihydroxyphenylalanine (DOPA).

According to some embodiments of the invention the DOPA is L-DOPA.

According to an aspect of some embodiments of the present invention there is provided a method of diagnosing a subject with skin cancer. The method comprises: (a) contacting at least one skin cell which is suspicious of a cancerous phenotype of the subject with L-DOPA or tyrosine under conditions wherein tyrosinase of the at least one cell catalyzes a reaction with the L-DOPA or tyrosine, so as to generate a product which produces an electrical signal; and (b) measuring a level of the electrical signal, wherein an increase in a strength of the electrical signal above a predetermined threshold is indicative of skin cancer, thereby diagnosing the subject with skin cancer.

According to an aspect of some embodiments of the present invention there is provided a method of individually optimizing a treatment for skin cancer. The method comprises: (a) contacting at least one skin cancer cell of a subject with at least one anti cancer agent; (b) contacting the at least one skin cancer cell with L-DOPA or tyrosine, under conditions wherein tyrosinase of the at least one cell catalyzes a reaction with the DOPA or tyrosine, so as to generate a product which produces an electrical signal; and (c) measuring a level of the electrical signal produced by the cell, wherein a decrease in the level is indicative of an efficient anti cancer agent for the treatment of the skin cancer of the subject, thereby individually optimizing a treatment for cancer.

According to an aspect of some embodiments of the present invention there is provided a method of monitoring an anti cancer treatment in a subject, the method comprising: (a) administering at least one anti cancer agent to the subject; and (b) detecting a presence or level of cancer cells in a sample of the subject According to some embodiments of the invention the presence or level is indicative of a state of the cancer, thereby monitoring an anti-cancer treatment in a subject.

According to some embodiments of the invention the skin cell is comprised in a skin tissue slice.

According to some embodiments of the invention the at least one skin cell is comprised in a skin tissue slice.

According to some embodiments of the invention the at least one skin cancer cell is comprised in a skin tissue slice.

According to some embodiments of the invention the sample comprises a skin tissue slice.

According to some embodiments of the invention the skin tissue slice is frozen prior to the contacting.

According to some embodiments of the invention the skin tissue slice is fixed by chemical fixatives prior to the contacting.

According to some embodiments of the invention the skin tissue slice is not pretreated prior to the contacting.

According to some embodiments of the invention the measuring is performed using means for high throughput.

According to some embodiments of the invention the means is selected from the group consisting of an automated sampling device, a liquid handling equipment, a dispenser, an electrode array, a robot, or any combination thereof.

According to some embodiments of the invention the contact is effected in vitro.

According to some embodiments of the invention the contact is effected ex vivo.

According to some embodiments of the invention the skin tissue slice comprises no more than one million cells.

According to some embodiments of the invention the skin tissue slice comprises no less than 10 cells.

According to some embodiments of the invention the measurement is effected using an electrochemical cell configured for sensing the produced signal.

According to some embodiments of the invention the measurement comprises: (i) establishing communication between the electrochemical cell and a measuring device configured for receiving and measuring an electrical signal generated by the electrochemical cell; and (ii) establishing communication between the measuring device and a hand-held electronic device supplemented by software for receiving, analyzing and presenting data pertaining to the measurement.

According to some embodiments of the invention the electrochemical cell and the measuring device are confined in the same physical encapsulation.

According to some embodiments of the invention the electrochemical cell and the measuring device are separated from each other and being in electrical communication thereamongst.

According to some embodiments of the invention the measuring is effected in a multiwell array.

According to some embodiments of the invention each well of the multiwell array comprises an electrochemical cell.

According to some embodiments of the invention each well of the multiwell array is a nano-volume well.

According to some embodiments of the invention the agent comprises a test composition.

According to some embodiments of the invention the test composition is selected from the group consisting of a polynucleotide a polypeptide, a small molecule chemical, a carbohydrate and a lipid.

According to some embodiments of the invention the agent comprises a test condition.

According to some embodiments of the invention the test condition is a radiation condition.

According to some embodiments of the invention the cells of the skin tissue slice are intact.

According to an aspect of some embodiments of the present invention there is provided a kit for determining a level of tyrosinase in a cell. The kit comprises: (i) L-DOPA or tyrosine; and (ii) an electrochemical cell.

According to some embodiments of the invention the L-DOPA or tyrosine is confined in the electrochemical cell.

According to some embodiments of the invention the L-DOPA or tyrosine and the electrochemical cell are in separate packaging.

According to an aspect of some embodiments of the present invention there is provided a system for detecting an analyte in a sample. The system comprises: electrochemical sensing system configured for receiving the sample, and generating an electrical signal responsively to the presence of the analyte therein; a measuring device, configured for receiving and measuring the electrical signal; and a hand-held electronic device being in communication with the measuring device and supplemented by software for receiving, analyzing and presenting data pertaining to the measurement.

According to some embodiments of the invention the hand-held electronic device is selected from the group consisting of a cellular telephone with data processing functionality, a personal digital assistant (PDA) with data processing functionality, a portable email device with data processing functionality, a portable media player with data processing functionality, a portable gaming device with data processing functionality, a tablet, and a touch screen display device with data processing functionality.

According to some embodiments of the invention the electrochemical sensing system and the measuring device are confined in the same physical encapsulation.

According to some embodiments of the invention the electrochemical sensing system and the measuring device are separated from each other and being in electrical communication thereamongst.

According to some embodiments of the invention the electrochemical sensing system is configured for receiving the sample in liquid form.

According to some embodiments of the invention the electrochemical sensing system is configured for receiving the sample in solid form.

According to some embodiments of the invention the sample is a skin tissue slice.

According to some embodiments of the invention the electrochemical sensing system comprises an electrochemical cell configured for detecting an analyte of a single and predetermined species.

According to some embodiments of the invention the electrochemical sensing system comprises a plurality of electrochemical cells, each being configured for detecting a different analyte.

According to some embodiments of the invention the analyte is selected from the group consisting of an environmental analyte, a clinical analyte, a chemical analyte, a pollutant, a biomolecule, a pesticide, a insecticide, a toxin, a therapeutic drug, an abused drug, a hormone and an antibiotic.

According to some embodiments of the invention the analyte comprises biomolecule selected from the group consisting of a polypeptide, a polynucleotide, a lipid, a carbohydrate, a steroid, a whole cell, a protein, an enzyme, an antibody, and antigen, a cellular membrane antigen, a receptor and a ligand.

According to some embodiments of the invention the analyte comprises a small molecule.

According to some embodiments of the invention the analyte comprises a genotoxic agent.

According to some embodiments of the invention the analyte comprises a biomarker.

According to some embodiments of the invention the biomarker is selected from the group consisting of Tyrosinase, IALP, ALP, LDH, CEA, CA15-3, PSA, IL-8, thioredoxin, thyroxine, PSA, CRP, alpha-fetoprotein, Enolase, prostatic acid phosphatase, BNP, PLGF, LH, Gelsolin, Perlecan, Lactoferrin, Orosomucoid, NMP22, Estrogen, Warfarin, Chloride, Troponin and Glycogen phosphorylase isoenzyme BB.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates how the substrate L-DOPA is oxidized by tyrosinase to product dopaquinone which is electroactive and hence is reduced by the working electrode in −300 mV.

FIG. 2 is a scheme depicting feasibility demonstration of the detection method. ‘Direct diagnosis’ was performed on slices of biopsies immediately upon their removal, suspended in an electrochemical cell, plugged to a potentiostat via a multiplexer. The substrate was added into the electrochemical cells and the resulting current measured.

FIG. 3 is a photograph of a tissue sample of ˜4 mm dissected from a biopsy removed from xenograft tumors. The tumors were induced in nude mice by injecting the melanoma cancer cell line, MEL526. Following mice sacrifice and collection of tumors, the dissected biopsy samples were put inside the electrochemical chamber, as described without pretreatment or further handling.

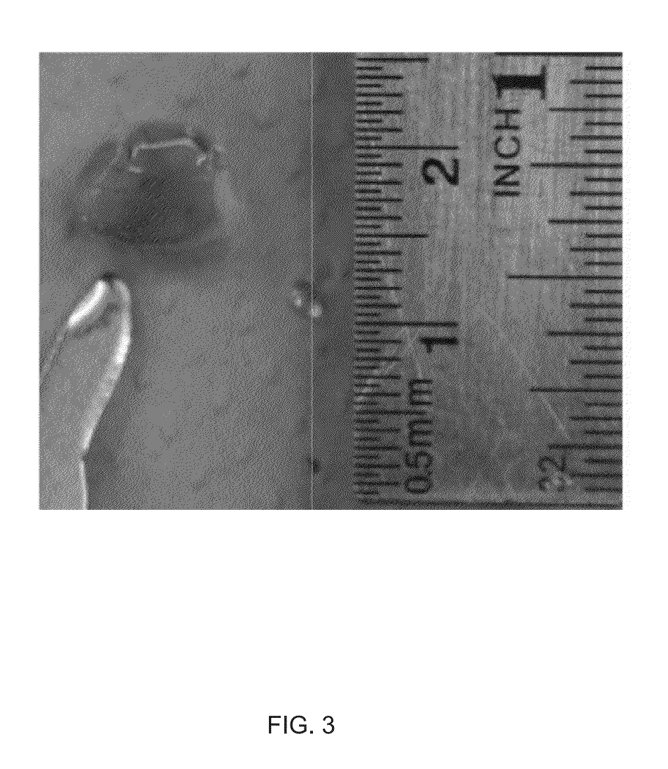

FIG. 4 is a graph illustrating the amperometric response of biopsies removed from MEL526 tumors versus healthy skin tissues. Current signals correspond to tyrosinase enzymatic activity.

FIG. 5 is a graph comparing MEL526 melanoma tissue with normal skin tissue tested. Results are represented as Δcurrent/Δtime (n=40), reflecting the slope of the generated current signals.

FIGS. 6A-C provide examples of electrochemical substrates utilized by a system according to some embodiments of the present invention. A) When the detected biomarker is ALP or when the enzyme label in ECI is ALP, the substrate used is pAPP (para-aminophenyl phosphate (or 1-naphthol). The substrate, p-aminophenyl phosphate is dephosphorylated by the enzyme ALP; the product p-aminophenol is oxidized on the electrode at 0.22 V, generating current. B) When HRP is the detected biomarker or when it is used as an enzyme label in ECI, the substrate used is APAP (acetaminophen). APAP is oxidized by HRP and H₂O₂ yielding the NAPQI (N-acetyl para benzoquinone imine) product which is than electrochemically reduced back under low potentials (around E=−80 mV). C) When the detected biomarker is the enzyme tyrosinase the substrate used is L-DOPA. This reagent is oxidized by tyrosinase to product dopaquinone which is electroactive and hence reduced by the working electrode in −300 mV.

FIGS. 7A-D is a scheme showing an example of direct electrochemical detection of enzymatic activity employed by a system according to some embodiments of the present invention. A) A meter or multi detector accommodates a multichip array (B) and the bio-specimen is contained within the chip chamber, in this example. C) A magnification of the chip chamber. An enzyme biomarker expressed by human cells indicating a disease or other disorder catalyzes the conversion of a specific substrate into an electroactive product which is than oxidized or reduced on the working electrode generating a measurable current detected as signal (D) by the potentiostat.

FIGS. 8A-E is a schematic description of the steps involved in the process of ECI. A) Antibodies specific against the selected biomarkers are immobilized onto the working electrode (WE). B) Human cells (from blood, body fluid, cell sample) are added and soluble biomarkers captured. C) HRP (horseradish peroxidase) labeled secondary antibody are added and bind the biomarker D) The HRP substrate is added. E) The modified biochips within the meter are subjected to an applied potential, allowing for the enzymatic electro-active product to be reduced/oxidized on the WE, generating measurable current.

FIG. 9 illustrates whole cell′ ECI for the detection of membrane bound biomarkers. Different cells are incubated with the anti-membrane biomarker antibody, and subsequently labeled cells are suspended with the HRP-labeled anti-human antibody. Chronoamperometry is initiated and upon substrate addition the resulting current is recorded.

FIG. 10 is a schematic illustration of a system for detecting an analyte in a sample, according to some embodiments of the present invention.

### DESCRIPTION OF SPECIFIC EMBODIMENTS OF THE INVENTION

The present invention, in some embodiments thereof, relates to a method of analyzing tyrosinase activity and, more particularly, but not exclusively, to use of same for detecting melanoma.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have developed a novel electrochemical method for sensitive and high-throughput detection of a cancer cell based on the level of expression of tyrosinase.

The results may be obtained within a few minutes from biopsy removal, since the cancerous cells are not required to be pre-treated prior to analysis.

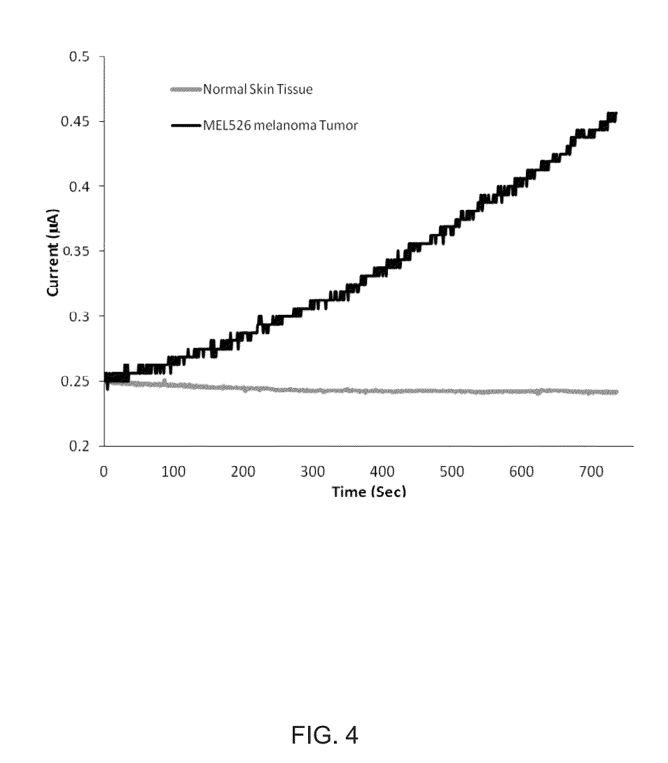

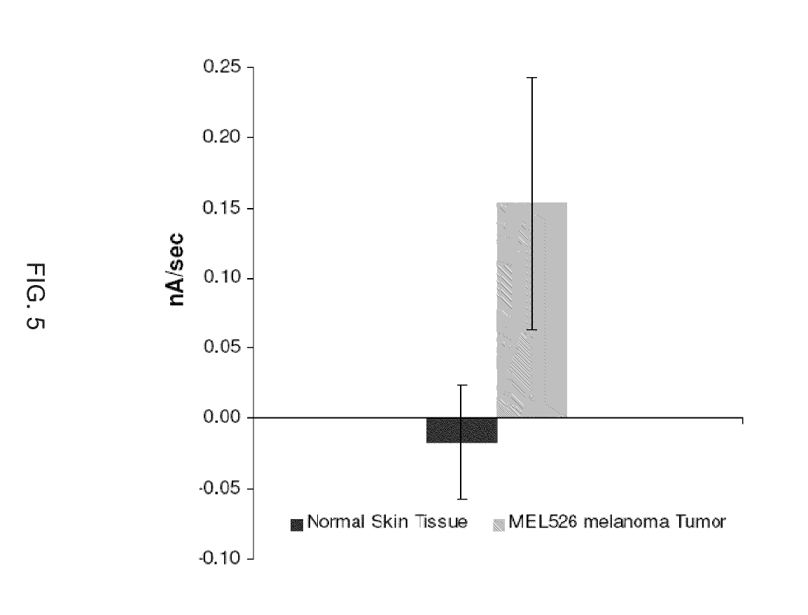

Whilst reducing the present invention to practice, the present inventors have shown that amperometric enzyme measurements may be performed with electrochemical substrates such as tyrosine or DOPA (e.g., L-DOPA) in order to detect melanoma cells. As illustrated in FIGS. 4 and 5, the proposed method could distinguish between healthy and cancerous cells in a highly sensitive, accurate and rapid fashion.

Multiple measurements yielded reproducible current signals thus supporting the feasibility of the biosensor and of the working hypothesis.

Thus, according to one aspect of the present invention there is provided a method of determining tyrosinase activity of a cell comprising:

(a) contacting the cell with a phenol under conditions wherein tyrosinase of the cell catalyzes a reaction with said phenol, so as to generate a product which produces an electrical signal; and

(b) measuring a level of said electrical signal, thereby determining tyrosinase activity of the cell.

As used herein, the term “cell” refers to a mammalian cell, preferably a human cell. Single cells may be used in accordance with the teachings of the present invention as well as plurality of cells. According to an exemplary embodiment, the plurality of cells comprises no less than 10 cells and no more than 500 cells. According to an exemplary embodiment, the plurality of cells comprises no less than 10 cells and no more than 100,000, 200,000, 500,000 or 1,000,000 cells. According to another exemplary embodiment the cells are in a single suspension such that the number of cells may be counted, although adherent cells and aggregates may still be detected.

According to another exemplary embodiment tissue slices are analyzed. The tissue slice may be as small as a cell aggregate, e.g., about 0.1 mm and as large as determined by the size of the device in which it is analyzed (e.g., a chamber of an electrochemical cell). Thus, typical sizes of the tissue slice are from about 0.1 mm to about 10 mm, or from about 0.5 mm to about 10 mm, or from about 1 mm to about 10 mm, or from about 1 mm to about 8 mm, or from about 1 mm to about 6 mm.

The plurality of cells may be from any biological sample such as cell-lines, primary cultures and cellular samples, e.g. biopsies (surgical biopsies including incisional or excisional biopsy, fine needle aspirates and the like), complete resections or body fluids. Methods of biopsy retrieval are well known in the art.

According to one embodiment, following biopsy removal, a tumor sample is sliced. In order to calibrate the system, such that comparison between healthy and non-healthy slices is accurate, the tumor slices may be weighed.

The cells in the biological sample may be assayed for tyrosinase enzyme activity with or without pretreatment. Thus, according to one embodiment, the cells in the biological sample are preferably intact (i.e. whole), and preferably viable.

According to another embodiment, the cells are fixed prior to analysis in a chemical fixative. An exemplary chemical fixative is a crosslinking fixative. Such fixatives include formaldehyde (e.g. a solution comprising 3-5% formaldehyde) and glutaraldehyde.

An exemplary fixative solution contemplated by the present invention is one which is a 10% Neutral Buffered Formalin (NBF), that is approximately 3.7% formaldehyde in phosphate buffered saline.

Other contemplated fixatives include precipitating fixatives, including, but not limited to ethanol, methanol and acetone; oxidizing agents, including but not limited to Osmium tetroxide, Potassium dichromate, chromic acid, and potassium permanganate; Mercurials such as B-5 and Zenker's; Picrates; Hepes-glutamic acid buffer-mediated organic solvent protection effect (HOPE).

Besides chemical fixation, the present invention also contemplates other forms of fixation such as frozen sections and heat fixation.

According to this aspect of the present invention, the cell sample may remain in the chemical fixative for one day, one week, one month, two months, three months or even longer. Typically, more than 50% of the enzymes in the cell sample are non-functional. According to another embodiment, more than 60% of the enzymes in the cell sample are non-functional. According to another embodiment, more than 70% of the enzymes in the cell sample are non-functional. According to another embodiment, more than 80% of the enzymes in the cell sample are non-functional. According to another embodiment, more than 90% of the enzymes in the cell sample are non-functional.

As mentioned hereinabove, the method of the present invention is effected by contacting an enzyme substrate (e.g. L-Dopa) with a cell, to bring about a reaction of the cell, wherein the product of the enzymatic reaction is capable of generating an electrical signal.

As used herein, the phrase “reaction of the cell” refers to a reaction that occurs between the substrate and an endogenous enzyme expressed by the cell, and not to a reaction that occurs with an exogenous enzyme.

As used herein, the term “contacting” refers to bringing the substrate into the vicinity of a cell under conditions such that the substrate may be catalyzed by the enzyme. Thus, for example, the contacting should be effected under buffer conditions, at a temperature and time sufficient to allow catalysis of the substrate and generation of sufficient product that it may be detected by an electrochemical cell. The contacting may be effected in vitro, ex vivo or in vivo. The contacting may be effected in a vessel which is also capable of detecting the product of the enzymatic reaction (i.e., in the electrochemical cell), such that the electrical signal is detected on-line. Such vessels are further described herein below. Alternatively, the contacting may be effected in a separate vessel from where the detection takes place such that it is possible to continuously withdraw samples at particular time points and place such samples within the electrochemical cells. Thus, the contacting may be effected in a test tube, flask, tissue culture, chip, array, plate, microplate, capillary, or the like. The cells may be placed on a vibrating plate following the addition of the substrate for continuous thorough mixing of the contents of the cells.

Contemplated substrates include the L-form of tyrosine or DOPA.

As mentioned hereinabove, electrochemical measurement of products capable of undergoing a redox reaction (i.e. capable of electron transfer) at an electrode of a chemical cell to yield an electrical signal (i.e. electrochemical products) is typically effected in electrochemical cells.

As used herein, the phrase “electrical signal” refers to electrons or electrochemically active species.

The phrase “electrochemical measurement” as used herein, refers to a measurement performed by the use of electrodes in a solution, typically in an electrochemical cell. The measurement may be performed, for example, by chronoamperometry, chrono-potentiometry, cyclic voltammetry, chrono-coulometry or square wave voltammetry. A signal detectable in such a measurement, is one that differs in such electrochemical measurement from the control.

The electrochemical cells of the present invention optionally comprise a working electrode, a counter electrode, a reference electrode and a chamber to hold the cells. In some embodiments of the present invention the electrochemical cells are configured for on-line measurement.

The working electrode may be of a variety of different kinds, for example, it may be made of carbon, including glassy carbon, activated carbon cloth electrode, carbon felt, platinized carbon cloth, plain carbon cloth), may be made of gold, platinum or silver. The counter electrode may also be made of the same material as the working electrode. The reference electrode may for example be saturated calomel electrode, may be an Ag/AgCl electrode. Furthermore, the electrodes may be of a screen printed electrode which can be inserted into the vessel comprising the cells without the need to withdraw a sample and transport it into a separate electrochemical cell.

The electrodes used to detect the product according to the method of the present invention may be reusable electrodes or disposable ones. Reusable electrodes may for example be electrodes made of glassy carbon in a disk or rod shape which are embedded in teflon. Disposable electrodes may for example be electrodes in the form of carbon paper, carbon cloth, carbon felts, or the screen printed electrode of the kind noted above.

According to one embodiment, the electrochemical cell is a three-electrode cell. According to another embodiment, the electrochemical cell is a two-electrode cell. According to a preferred embodiment the electrochemical cells are provided as an array (i.e. chip) comprising a plurality of such cells i.e. a multiwell array where each well is of a nano-volume size.

The system for measuring the electrical signal generated by the reaction product may further comprise a control module which may be a computer, a potentiostat and a multiplexer module which is needed in case of a typical embodiment for simultaneous measurement from a plurality of electrochemical cells.

The electrochemical measurement performed in the cell will now be described in reference to the chrono-amperometric mode. As will be appreciated, it applies, mutatis, mutandis also to the other electrochemical measurement modes mentioned above. Furthermore, the description will be made with reference to the use of a multi-electrode system (the system comprising an array of electrodes) and it is clear that it applies to a system comprising a single cell as well.

In the beginning of the electrochemical measurement all the electrodes are operated together, and the computer scans all the electrodes via the parallel port, and the background response to the potential application of each electrode is recorded by the computer. The entire electrochemical measurement sequence can be performed over a long period of time while measuring the currents resulting from the changes in the concentration of the products. In cases where the electrodes' surfaces are not identical due to natural variability, the system can be calibrated by measuring the oxidation or reduction of an electroactive species, typically the same species which is the product of the enzymatic reaction in the electrochemical cell and comparison of the results of all the electrodes.

In performing the assay, the electrodes may be connected to the potentiostat and at the same time also collected via the multiplexer to a parallel port of the microcomputer.

The electrochemical cell can contain a reference electrode and a counter electrode which can also be connected to the potentiostat. A specific potential is applied by the potentiostat on the electrodes (which can be the same for all the electrodes or can be a different potential to each electrode) and the current in each electrode is detected. The electrical signals are visualized, optionally and preferably, in real-time, on the computer screen.

Additional biological sensors suitable for the present embodiments are described hereinbelow.

Since the electrical signals generated by the electrochemical products of the enzymatic reactions reflect the level of tyrosinase in the cell, and tyrosinase (presence, absence or level of same) is a marker for particular cancers, the signals may be used to determine whether a cell is cancerous (i.e. malignant) or not. Specifically, if the level of the generated electrical signal is different to a predetermined threshold, this would indicate that the cell is cancerous. Typically, the predetermined threshold is determined by the electrical signal generated by a control cell.

A “cancer cell”, also referred to herein as a “malignant cell”, is a cell which has been released from normal cell division control, and is thus characterized by an abnormal growth and a tendency to proliferate in an uncontrolled way and, in some cases, to metastasize. Accordingly, the cancer cell may be a neoplastic cell, a pre-malignant cell, a metastatic cell, a tumor cell, an oncogenic cell, a cell with a cancer genotype, a cell of malignant phenotype, an oncogene transfected cell, a virus transformed cell, a cell which expresses an oncogene, a cell which expresses a marker for cancer, or a combination thereof.

Non-limiting examples of a cancer cell which may be detected by the method of the present invention is: an adenocarcinoma cell, an adrenal gland tumor cell, an ameloblastoma cell, an anaplastic cell, anaplastic carcinoma of the thyroid cell, an angiofibroma cell, an angioma cell, an angiosarcoma cell, an apudoma cell, an argentaffmoma cell, an arrhenoblastoma cell, an ascites tumor cell, an ascitic tumor cell, an astroblastoma cell, an astrocytoma cell, an ataxia-telangiectasia cell, an atrial myxoma cell, a basal cell carcinoma cell, a benign tumor cell, a bone cancer cell, a bone tumor cell, a brainstem glioma cell, a brain tumor cell, a breast cancer cell, a Burkitt's lymphoma cell, a cancerous cell, a carcinoid cell, a carcinoma cell, a cerebellar astrocytoma cell, a cervical cancer cell, a cherry angioma cell, a cholangiocarcinoma cell, a cholangioma cell, a chondroblastoma cell, a chondroma cell, a chondrosarcoma cell, a chorioblastoma cell, a choriocarcinoma cell, a colon cancer cell, a common acute lymphoblastic leukemia cell, a craniopharyngioma cell, a cystocarcinoma cell, a cystofbroma cell, a cystoma cell, a cytoma cell, a ductal carcinoma in situ cell, a ductal papilloma cell, a dysgerminoma cell, an encephaloma cell, an endometrial carcinoma cell, an endothelioma cell, an ependymoma cell, an epithelioma cell, an erythroleukemia cell, an Ewing's sarcoma cell, an extra nodal lymphoma cell, a feline sarcoma cell, a fibro adenoma cell, a fibro sarcoma cell, a follicular cancer of the thyroid cell, a ganglioglioma cell, a gastrinoma cell, aglioblastoma multiform cell, a glioma cell, a gonadoblastoma cell, an haemangioblastomacell, an haemangioendothelioblastoma cell, an haemangioendothelioma cell, an haemangiopericytoma cell, an haematolymphangioma cell, an haemocytoblastoma cell, an haemocytoma cell, a hairy cell leukemia cell, a hamartoma cell, an hepatocarcinoma cell, an hepatocellular carcinoma cell, an hepatoma cell, an histoma cell, a Hodgkin's disease cell, an hypernephroma cell, an infiltrating cancer cell, an infiltrating ductal cell carcinoma cell, an insulinoma cell, a juvenile angioforoma cell, a Kaposi sarcoma cell, a kidney tumor cell, a large cell lymphoma cell, a leukemia cell, a chronic leukemia cell, an acute leukemia cell, a lipoma cell, a liver cancer cell, a liver metastases cell, a Lucke carcinoma cell, a lymphadenoma cell, a lymphangioma cell, a lymphocytic leukemia cell, a lymphocytic lymphoma cell, a lymphoeytoma cell, a lymphoedema cell, a lymphoma cell, a lung cancer cell, a malignant mesothelioma cell, a malignant teratoma cell, a mastocytoma cell, a medulloblastome. cell, a melanoma cell, a meningioma cell, a mesothelioma cell, a metastatic cell, a metastasis cell, a metastatic spread cell, a Morton's neuroma cell, a multiple myeloma cell, a myeloblastoma cell, a myeloid leukemia cell, a myelolipoma cell, a myeloma cell, a myoblastoma cell, a myxoma cell, a nasopharyngeal carcinoma cell, a neoplastic cell, a nephroblastoma cell, a neuroblastoma cell, a neurofibroma cell, a neurofibromatosis cell, a neuroglioma cell, a neuroma cell, a non-Hodgkin's lymphoma cell, an oligodendroglioma cell, an optic glioma cell, an osteochondroma cell, an osteogenic sarcoma cell, an osteosarcoma cell, an ovarian cancer cell, a Paget's disease of the nipple cell, a pancoast tumor cell, a pancreatic cancer cell, a phaeochromocytoma cell, a pheoehromocytoma cell, a plasmacytoma cell, a primary brain tumor cell, a progonoma cell, a prolactinoma cell, a renal cell carcinoma cell, a retinoblastoma cell, a rhabdomyosarcoma cell, a rhabdosarcoma cell, a solid tumor cell, sarcoma cell, a secondary tumor cell, a seminoma cell, a skin cancer cell, a small cell carcinoma cell, a squamous cell carcinoma cell, a strawberry haemangioma cell, a T-cell lymphoma cell, a teratoma cell, a testicular cancer cell, a thymoma cell, a trophoblastic tumor cell, a tumorigenic cell, a tumor initiation cell, a tumor progression cell, a vestibular schwannoma cell, a Wilm's tumor cell, or a combination thereof.

According to a preferred embodiment of this aspect of the present invention, the cancer cell is a skin cancer cell.

Examples of skin cancer cells include basal cell carcinoma, squamous cell carcinoma and malignant melanoma.

Additional examples of skin cancers include Dermatofibrosarcoma protuberans, Merkel cell carcinoma, Kaposi's sarcoma, keratoacanthoma, spindle cell tumors, sebaceous carcinomas, microcystic adnexal carcinoma, Pagets's disease of the breast, atypical fibroxanthoma, leimyosarcoma, and angiosarcoma.

The control cell can be a normally differentiated cell, non-cancerous cell, preferably of the same tissue and specimen as the tested cell suspicious of a cancerous or undifferentiated phenotype. Preferably, the difference is at least 10%, 20%, 30%, 40%, 50%, 80%, 100% (i.e., two-fold), 3 fold, 5 fold or 10 fold different as compared to a control cell.

According to another embodiment of the present invention, the amount of enzyme (and accordingly electrical signal) in a cancer cell is higher than the amount of enzyme (and accordingly electrical signal) in a non-cancer cell.

It will be appreciated that the method of the present invention may be used for diagnosing a subject with cancer.

As used herein the term “diagnosing” refers to classifying a cancer, determining a severity of cancer (grade or stage), monitoring cancer progression, forecasting an outcome of the cancer and/or prospects of recovery.

The subject may be a healthy animal or human subject undergoing a routine well-being check up. Alternatively, the subject may be at risk of having cancer (e.g., a genetically predisposed subject, a subject with medical and/or family history of cancer, a subject who has been exposed to carcinogens, occupational hazard, environmental hazard] and/or a subject who exhibits suspicious clinical signs of cancer [e.g., blood in the stool or melena, unexplained pain, sweating, unexplained fever, unexplained loss of weight up to anorexia, changes in bowel habits (constipation and/or diarrhea), tenesmus (sense of incomplete defecation, for rectal cancer specifically), anemia and/or general weakness).

Although the present invention can, in theory, be practiced with a single electrochemical cell, such a method is not efficient nor is it desirable. Preferably, the method of the present invention is used for high throughput screening of agents using a plurality of electrochemical cells to simultaneously screen a variety of agents. The cells may be part of a chip, for example a silicon chip.

Thus, according to one embodiment, the method of the present invention is performed using means for high throughput. Accordingly, the method may be performed, for example, using an automated sampling device, a liquid handling equipment, a dispenser, an electrode array, a robot, or any combination thereof.

It will be appreciated that the present has a variety of applications pertaining to individually optimizing a treatment for cancer, monitoring an-anti cancer treatment in a subject, determining an anti cancer treatment for a subject and identifying an agent capable of reversing a malignant phenotype of a cell.

Thus, according to another aspect of the present invention, there is provided a method of identifying an agent capable of reversing a malignant phenotype of a cell. The method comprises subjecting at least one cancer cell to an agent and determining the efficiency of the anti cancer agent by monitoring the activity or expression tyrosinase according to the method of the present invention.

As used herein the phrase “reversing a malignant phenotype” refers to at least partially reversing the proliferative and/or invasive characteristics of the malignant cell.

As used herein, the term “agent” refers to a test composition comprising a biological agent or a chemical agent.

Examples of biological agents that may be tested as potential anti cancer agents according to the method of the present invention include, but are not limited to, nucleic acids, e.g., polynucleotides, ribozymes, siRNA and antisense molecules (including without limitation RNA, DNA, RNA/DNA hybrids, peptide nucleic acids, and polynucleotide analogs having altered backbone and/or bass structures or other chemical modifications); proteins, polypeptides (e.g. peptides), carbohydrates, lipids and “small molecule” drug candidates. “Small molecules” can be, for example, naturally occurring compounds (e.g., compounds derived from plant extracts, microbial broths, and the like) or synthetic organic or organometallic compounds having molecular weights of less than about 10,000 daltons, preferably less than about 5,000 daltons, and most preferably less than about 1,500 daltons.

Examples of conditions that may be tested as potential anti cancer agents according to the method of the present invention include, but are not limited to, radiation exposure (such as, gamma radiation, UV radiation, X-radiation).

According to an embodiment of this aspect of the present invention, the “tyrosinase enzyme” is also assayed prior to contact with the agent so that a comparison may be made prior to and following treatment.

According to another embodiment of this aspect of the present invention, the agent is subjected to the cancer cells for a period long enough to have an anti cancer effect. Thus, for example if dacarbazine and/or temozolomide are being analyzed, preferably these agents are subjected to the cancer cells for at least 1 day and more preferably 3 days.

It will be appreciated that the agent may be contacted with cancer cells either in vitro, ex vivo or in vivo. If the contacting is effected in vivo, the cells are typically removed from the subject prior to contact with the substrate of the present invention.

The present invention can, in theory, be practiced with a single electrochemical cell. Preferably, the method of the present invention is used for high throughput screening of agents using a plurality of electrochemical cells to simultaneously screen a variety of agents. The cells may be part of a chip, for example a silicon chip as described in U.S. Pat. No. 8,268,577, incorporated herein by reference.

Thus, according to one embodiment, the method of the present invention is performed using means for high output. Accordingly, the method may be performed, for example, using an automated sampling device, a liquid handling equipment, a dispenser, an electrode array, a robot, or any combination thereof.

It is now known that tumor treatment response cannot be predicted only from its type and anatomical location. It will be appreciated that the method of identifying an agent capable of reversing a malignant phenotype of a cell may be modified such that particular patient's cells may be used in the assay system, thereby tailoring therapeutic agents to specific patients. Furthermore, it will be appreciated that not only may the specific agent be selected using the method of the present invention, but the optimal dose and optimal treatment regimen may also be identified according to the method of the present invention. In this way a therapeutically effective amount of an agent may be determined.

The patient may be treated according to the optimal treatment conditions selected with the aid of the method of the present invention and optionally retested after a suitable time period. In this way a patient's response may be continually monitored whilst undergoing therapy.

Conceivably the analyzing tyrosinase levels and administering steps may be repeated a number of times during the course of a treatment. For instance the tyrosinase levels may be analyzed one week following administration of the agent. If the tyrosinase levels are higher than those compared with a control, the dose of the agent may be increased.

It will be appreciated that the electrochemical cell of the present invention may be provided in a kit together with at least one anti-cancer agent for determining an effect thereof on a cancer cell. The kit of the present invention may, if desired, be presented in a pack which may contain one or more units of the kit of the present invention. The pack may be accompanied by instructions for using the kit and the estimated dose of the anti-cancer agent for a particular number of cells. The pack may also be accommodated by a notice associated with the container in a form prescribed by a governmental agency regulating the manufacture, use or sale of laboratory supplements, which notice is reflective of approval by the agency of the form of the compositions.

According to one embodiment, the kit may also comprise a substrate which is enzymatically reacted on by the tyrosinase of the biological cell (i.e. cancer cell) to yield a reaction product giving rise to a redox reaction at an electrode of the electrochemical cell—including for example L-DOPA or tyrosine. Such substrates have been described herein above.

As mentioned herein above the present invention contemplates the use of one or more electrochemical cells for sensing the signal.

According to some embodiments of the present invention communication is established between the electrochemical cell and a measuring device configured for receiving and measuring an electrical signal generated by the electrochemical cell. The measuring device optionally and preferably communicates with a data processor supplemented by software for receiving, analyzing and presenting data pertaining to the measurement.

As used herein, “data processor” includes any suitable device for processing data, including, without limitation, a microcomputer, a microprocessor, and a data processing system. A data processor can be electronic computing circuitry (e.g., a central processing unit) or a system associated with such circuitry. Representative examples include, without limitation, a desktop home computer, a workstation, a laptop computer and a notebook computer. Also contemplated is a dedicated system having electronic computing circuitry therein. Optionally, such a dedicated system is portable. Optionally, such a dedicated system is hand held or wearable, e.g., on the arm of the user. Also contemplated are systems which are capable of receiving and processing data but may also have other functions. Representative examples include, without limitation, a cellular telephone with data processing functionality (also known as a smartphone), a personal digital assistant (PDA) with data processing functionality, a portable email device with data processing functionality (e.g., a BlackBerry® device), a portable media player with data processing functionality (e.g., an Apple iPod®), a portable gaming device with data processing functionality (e.g., a Gameboy®), and a tablet or touch screen display device with data processing functionality (e.g., an Apple iPad®, the Motorola Xoom®, Samsung Galaxy®, and the TabletKiosk Sahara NetSlate®).

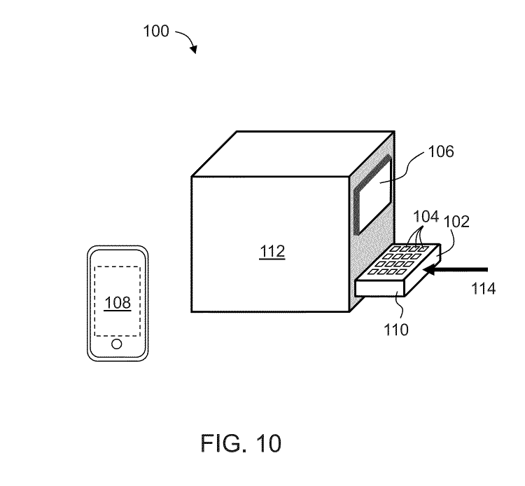

Thus, referring to FIG. 10, the present embodiments contemplate a system 100 comprising an electrochemical system 102 having one or more electrochemical cells 104, a measuring device 106 and an electronic device 108, such as a data processor as further detailed hereinabove.

The communication between the data processor and the measuring device is optionally and preferably by electronic signal is transmitted through an interface such as, but not limited to, an IEEE 1394 interface, a USB interface, a wireless interface and the like. Wireless interface may feature, for example, Bluetooth communication, IEEE 802.11(b) (WiFi) communication, Wi-Max communication, or wireless USB communication.

The data processor is preferably supplemented by software programmed for receiving electrical signals from the measuring device, analyzing the signal and presenting an output pertaining to the analysis. The software is optionally and preferably also designed for providing a virtual user interface, e.g., by means of a tough or multi-touch screen, so as to allow the user to interact with the data processor.

The electrochemical system of the present embodiments preferably comprises one or more electrochemical cells 104, optionally and preferably microchambers, formed on a substrate 110 which can be a generally planar substrate, e.g., a silicon wafer or the like. Each of the electrochemical cells can comprise several electrodes. For example, electrochemical cells can comprise a working electrode, a counter electrode and a reference electrode, as further detailed hereinabove.

The electrochemical system can be fabricated using any known microelectronic fabrication technique, particularly, but not exclusively, processes suitable for microelectromechanical systems (MEMS). The fabrication process can be a subtractive process, an additive process or a combined process which includes a combination of subtractive steps and additive steps. Thus, the fabrication process includes at least one of: photolithography, evaporation, deposition, etching (using either wet chemical processes or plasma processes), focused ion milling, and lift off.

The walls of the electrochemical cell(s) can be made of any material suitable for microelectronic applications, such as, but not limited to, a photoresist material or the like, that can be spun onto the substrate. A representative example of a photoresist material suitable for the present embodiments includes, without limitation, SU-8 (e.g., SU-8 3050) that is currently manufactured and sold by MicroChem Corporation.

The electrodes are preferably made, at least in part from a metal or a metal alloy, such as, but not limited to, gold, silver, copper and any combination thereof. Coated and modified electrodes are also contemplated. The reference electrode is optionally and preferably coated by a combination of materials selected from the group consisting of silver/silver chloride, silver/silver bromide, silver/silver fluoride, and silver/silver iodide, copper/copper halide, copper/copper oxide, copper/copper sulfate and the like, as known in the art. In some embodiments of the present invention, the working electrode is coated by a conductive polymer, such as, but not limited to, polypyrrole, polyaniline, polythiophene and polyacetylene.

The electrodes can be planar or they can have any other geometrical shape.

A “planar electrode,” as used herein, refers to an electrode which projects upwardly from a base of the microchamber, by less than one micron or less than 500 nm or less than 400 nm.

In some embodiments, each microchamber comprises at least one or at least two or at least three planar electrodes. A representative example includes a configuration in which the electrochemical cell has a planar working electrode, a planar counter electrode and a planar reference electrode. Typically, but not necessarily, the height of the planar reference electrode is higher by about 500 nm than the heights of the planar working electrode and the planar counter electrode. Thus, for example, the planar reference electrode can has a height of about 800 nm, and each of the planar working electrode and the planar counter electrode can has a height of about 300 nm.

In some embodiments, the working electrode is generally shaped as a pillar projecting upwardly from the base of microchamber. These embodiments are particularly useful when it is desired to increase the sensing area of the working electrode. In some embodiments of the present invention the height of working electrode above the base is at least 10 times higher than the heights of the electrodes.

The electrodes area is dependent upon the specimen volume. A typical surface area of the electrodes is from about 0.0.1 mm² to about 4 mm². The system configuration which is intended for home use optionally and preferably employs a biochip with an additional reaction chamber and on-chip integrated biology.

In some embodiments of the invention the electrochemical cell comprises a biological sensor which produces an electrochemical signal in the electrochemical cell. The biological sensor preferably generates a signal in response to presence of a particular substance or a particular family or group of substances or some particular substances or families or groups of substances in the liquid. For example, the biological sensor can comprise a cell capable of reporter expression when the cell is exposed to an analyte of interest.

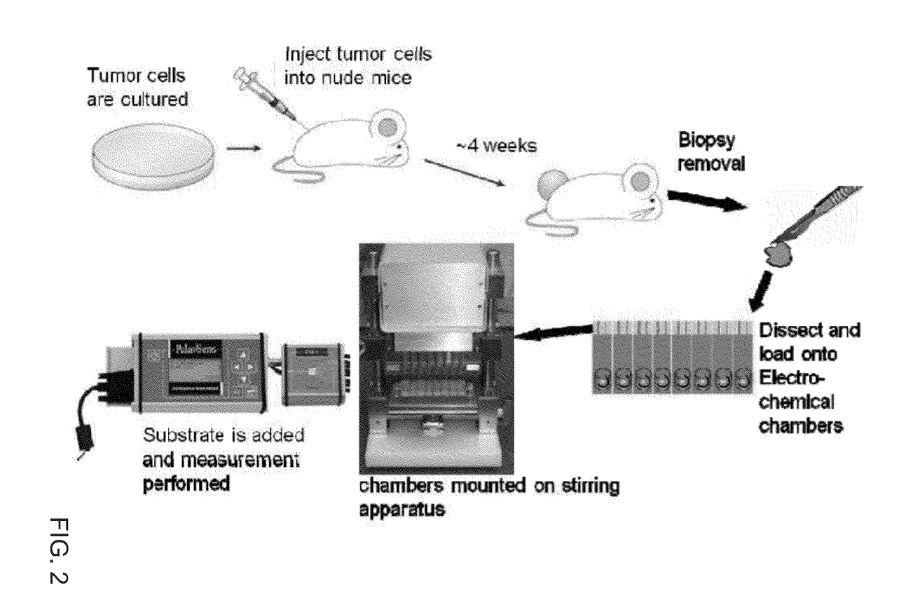

The electrochemical cell(s) and the measuring device can be confined in the same physical encapsulation, or they can be separated from each other. In the schematic illustration of FIG. 10, which is not to be considered as limiting, the measuring device 106 is encapsulated in a physical encapsulation 112 configured for receiving substrate 110 with cells 104, as illustrated by arrow 114. In any event, the electrochemical cell(s) and the measuring device communicate with each other so as to allow the measuring device to receive the signals from electrochemical cell(s). When the electrochemical cell(s) and the measuring device are confined in the same physical encapsulation, the electrochemical cell(s) and optionally also the measuring device are detachable from the encapsulation. In use, the sample can be loaded into the electrochemical cell(s) which can subsequently be mounted onto the encapsulation as schematically illustrated, for example, in FIG. 2. Signal exchange between the electrochemical cell(s) and the measuring device as well as between the measuring device and electronic device or data processor can be triggered upon mounting or responsively to user input, as desired.

It will be appreciated that a system comprising one or more electrochemical cells, a measuring device and an electronic device as further detailed hereinabove, may be used for detecting the level of additional analytes other than tyrosinase.

As used herein, the term “analyte” refers to a molecule or compound to be detected. Suitable analytes include organic and inorganic molecules, including biomolecules. The analyte may be an environmental or clinical chemical or pollutant or biomolecule, including, but not limited to, pesticides, insecticides, toxins, therapeutic and abused drugs, hormones, antibiotics, organic materials, and solvents. Suitable biomolecules include, but are not limited to, polypeptides, polynucleotides, lipids, carbohydrates, steroids, whole cells including prokaryotic (such as pathogenic bacteria) and eukaryotic cells, including mammalian tumor cells, viruses, spores, etc. Particularly preferred analytes are proteins including enzymes; drugs, antibodies; antigens; cellular membrane antigens and receptors (neural, hormonal, nutrient, and cell surface receptors) or their ligands.

Other examples of analytes include, but are not limited to, small molecules such as naturally occurring compounds (e.g., compounds derived from plant extracts, microbial broths, and the like) or synthetic compounds having molecular weights of less than about 10,000 daltons, preferably less than about 5,000 daltons, and most preferably less than about 1,500 daltons, electrolytes, metals, peptides, nucleotides, saccharides, fatty acids, steroids and the like. Analytes typically include at least one functional group necessary for biological interactions (e.g., amine group, carbonyl group, hydroxyl group, carboxyl group).

According to some embodiments, the analyte is a genotoxic agents i.e., a genotoxicant.

As used herein, the term “genotoxicant” refers to a chemical, physical or biological agent that damages the DNA of a cell.

The genotoxicant may cause damage which is manifested by halting of DNA synthesis (e.g., antibiotic e.g., nalidixic acid (NA)), DNA cross-linking, DNA breaks and the like.

According to a specific embodiment, the genotoxicant is mitomycin C.

According to a specific embodiment, the genotoxicant is H₂O₂.

According to a specific embodiment, the genotoxicant is nalidixic acid.

According to a specific embodiment, the genotoxic agent is a chemotherapy.

Genotoxic chemotherapy may be divided into alkylating agents (i.e., drugs that modify the bases of DNA, interfering with DNA replication and transcription and leading to mutations); intercalating agents (i.e., drugs that wedge themselves into the spaces between the nucleotides in the DNA double helix. They interfere with transcription, replication and induce mutations); and enzyme inhibitors (i.e., drugs that inhibit key enzymes, such as topoisomerases, involved in DNA replication inducing DNA damage).

The goal of treatment with any of these agents is the induction of DNA damage in the cancer cells. DNA damage, if severe enough, will induce cells to undergo apoptosis, the equivalent of cellular suicide. The genotoxic chemotherapy drugs affect both normal and cancerous cells. The selectivity of the drug action is based on the sensitivity of rapidly dividing cells, such as cancer cells, to treatments that damage DNA. The mode of action also explains many of the side effects of treatment with these drugs. Rapidly dividing cells, such as those that line the intestine or the stem cells in bone marrow, are often killed along with the cancer cells. In addition to being cytotoxic (cell poisons), these drugs are also mutagenic (cause mutations) and carcinogenic (cause cancer). Treatment with these drugs carries with it the risk of secondary cancers, such as leukemia. These drugs are used to treat a variety of solid cancers and cancers of blood cells, often in combination with other drugs. Specific examples of chemotherapeutic genotoxicants include, but are not limited to, Busulfan, Bendamustine, Carboplatin, Carmustine, Chlorambucil, Cisplatin, Cyclophosphamide, Dacarbazine, Daunorubicin, Decitabine, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide and Topotecan.

According to another embodiment the analyte is a biomarker (i.e. correlates with a disease).

The series of functional biomarkers which may be detected by the system of the present embodiments covers a wide range of potential applications. The following list, presented in Table 1, includes representative and non-limiting examples of some of the biomarkers which may be detected by the system. Also shown, is the related pathology or disease indicator for each marker, the method used by the system, the type and origin of the biospecimen, the application category and the utilized substrate for detection.

TABLE 1

| biomarker | pathology/disease | specimen | | application | |
|---|---|---|---|---|---|
| measured | indicator | type | method | category | substrate |
| Tyrosinase | Melanoma/melanocyte | Tissue | Direct | Clinical use | L-dopa |
| | differentiation marker | (skin), | | (sln, mohs, | |
| | | blood | | dermatologist | |
| | | | | site) | |
| IALP (intestinal | CRC/intestinal | Tissue | Direct | Clinical use | pAPP, 1- |
| alkaline | differentiation marker | (epithelial | | (colonoscopy, | Naphthol |
| phosphatase) | | colon) | | intraoperative) | |
| ALP (general) | Liver damage, bone | Blood | Direct | Home use and | pAPP, 1- |
| | disease, | | | clinical setting | Naphthol |
| | hyperparathyroidism, | | | | |
| | vitamin D deficiency, | | | | |
| | hepatobiliary system | | | | |
| | etc. | | | | |
| LDH (lactate | tissue | blood | direct | home use and | Lactate, |
| dehydrogenase) | damage/breakdown, | | | clinical setting | NAD+ |
| | liver disease, heart | | | | |
| | attack, anemia, muscle | | | | |
| | trauma, bone | | | | |
| | fractures, cancers, and | | | | |
| | infections such as | | | | |
| | meningitis, | | | | |
| | encephalitis, and HIV | | | | |
| CEA | colorectal carcinoma, | Blood, | Immunoassay | clinical setting | enzyme |
| (carcinoembryonic | gastric carcinoma, | tissue | | | label |
| antigen) | pancreatic carcinoma, | | | | dependent |
| | lung carcinoma, breast | | | | (usually |
| | carcinoma, medullary | | | | APAP) |
| | thyroid carcinoma, | | | | |
| | ulcerative colitis, | | | | |
| | pancreatitis, cirrhosis, | | | | |
| | COPD, Crohn's | | | | |
| | disease | | | | |
| CA19-9 | pancreatic cancer, | Blood, | Immunoassay | clinical setting | enzyme |
| | colorectal, lung, and | tissue | | | label |
| | gall bladder cancers, | | | | dependent |
| | gall stones, | | | | (usually |
| | pancreatitis, cystic | | | | APAP) |
| | fibrosis, and liver | | | | |
| | disease. | | | | |
| MUC5AC | colorectal adenomas | tissue, | Immunoassay | clinical | enzyme |
| | and carcinomas, | blood, | | setting, home | label |
| | gallbladder | mucus | | use | dependent |
| | adenocarcinoma, | | | | (usually |
| | Gastric cancer, | | | | APAP) |
| | Endometrial | | | | |
| | adenocarcinoma, | | | | |
| | Pancreatic cancer, | | | | |
| | Airways pathologies: | | | | |
| | asthma, cystic fibrosis | | | | |
| | (CF), chronic | | | | |
| | obstructive pulmonary | | | | |
| | disease (COPD) and | | | | |
| | nasal polyps (NP) in | | | | |
| | upper airways | | | | |
| EGFR (Epithelial | breast, colon, | tissue | Immunoassay | clinical setting | enzyme |
| growth factor | epidermoid | | | | label |
| receptor) | carcinomas | | | | dependent |
| | | | | | (usually |
| | | | | | APAP) |
| CA-125 | ovarian, endometrial, | blood | Immunoassay | clinical setting | enzyme |
| | fallopian tube, lung, | | | | label |
| | breast and | | | | dependent |
| | gastrointestinal | | | | (usually |
| | cancers, | | | | APAP) |
| | endometriosis, several | | | | |
| | diseases of the ovary, | | | | |
| | abdominal | | | | |
| | inflammation | | | | |
| CA15-3 | benign breast or | blood | Immunoassay | clinical setting | enzyme |
| | ovarian disease, | | | | label |
| | endometriosis, pelvic | | | | dependent |
| | inflammatory disease, | | | | (usually |
| | and hepatitis, cancers | | | | APAP) |
| | of the ovary, lung, and | | | | |
| | prostate, effect of | | | | |
| | treatment for breast | | | | |
| | cancer. | | | | |
| PSA | Prostate cancer | blood | Immunoassay | clinical setting | enzyme |
| | | | | | label |
| | | | | | dependent |
| | | | | | (usually |
| | | | | | APAP) |
| IL-8 | inflammation marker, | blood, | Immunoassay | clinical setting | enzyme |
| | gingivitis, psoriasis, | saliva | | | label |
| | oral cancers | | | | dependent |
| | | | | | (usually |
| | | | | | APAP) |
| thioredoxin | Acute Myocardial | blood | direct | clinical | |
| | Infarction, various | | | setting, home | |
| | oxidative stresses | | | use | |
| | including ultraviolet | | | | |
| | rays, radiation, | | | | |
| | oxidants, viral | | | | |
| | infections, ischemia | | | | |
| | reperfusion or | | | | |
| | anticancer agents | | | | |
| thyroxine | thyroid disease | blood | direct or | home use, | |
| | | | immunoassay | clinical setting | |
| PSA | prostate cancer | blood | | | |
| CRP | acute inflammation | blood | immunoassay | clinical setting | enzyme |
| | such as infection, | | | | label |
| | renal failure, | | | | dependent |
| | atherosclerotic | | | | (usually |
| | disease, stroke, | | | | APAP) |
| | myocardial infarction, | | | | |
| | and severe peripheral | | | | |
| | vascular disease. | | | | |
| alpha-fetoprotein | liver cancer or cancer | blood | immunoassay | clinical setting | enzyme |
| (AFP) | of the ovary or testicle | | | | label |
| | | | | | dependent |
| | | | | | (usually |
| | | | | | APAP) |
| Enolase | low-grade | body | direct | clinical setting | |
| | astrocytoma, | fluid | | | |
| | neuroblastoma, | (CSF) | | | |
| | treatment monitoring | | | | |
| | in small cell lung | | | | |
| | cancer | | | | |
| prostatic acid | prostate cancer, | blood | direct | clinical setting | pAPP, |
| phosphatase | testicular cancer, | | | or home use | phenyl |
| (PAP) | leukemia, and non- | | | | phosphate, |
| | Hodgkin's lymphoma, | | | | 1- |
| | Diseases of the bone, | | | | Naphthol |
| | such as Paget's disease | | | | |
| | or | | | | |
| | hyperparathyroidism, | | | | |
| | diseases of blood | | | | |
| | cells, such as sickle- | | | | |
| | cell disease or | | | | |
| | multiple myeloma or | | | | |
| | lysosomal storage | | | | |
| | diseases, such as | | | | |
| | Gaucher's disease, | | | | |
| | presumptive test for | | | | |
| | semen | | | | |
| BNP | Heart failure | Blood | Immunoassay | Clinical | Enzyme |
| | | | | setting or | label |
| | | | | home use | dependent |
| | | | | | (usually |
| | | | | | APAP) |
| PLGF | Pre-eclampsia | Blood | Immunoassay | Clinical | Enzyme |
| | | | | setting or | label |
| | | | | home use | dependent |
| | | | | | (usually |
| | | | | | APAP) |
| LH | Ovulation | Urine | Immunoassay | Home use | Enzyme |
| | | | | | label |
| | | | | | dependent |
| | | | | | (usually |
| | | | | | APAP) |
| Gelsolin | Obstructive sleep | Urine | Immunoassay | Home use | Enzyme |
| | apnea | | | | label |
| | | | | | dependent |
| | | | | | (usually |
| | | | | | APAP) |
| Perlecan | Obstructive sleep | Urine | Immunoassay | Home use | Enzyme |
| | apnea | | | | label |
| | | | | | dependent |
| | | | | | (usually |
| | | | | | APAP) |
| Lactoferrin | Urinary tract infection | Urine | Immunoassay | Home use | Enzyme |
| | | | | | label |
| | | | | | dependent |
| | | | | | (usually |
| | | | | | APAP) |
| Orosomucoid | Pre-eclampsia | Urine | Immunoassay | Home use | Enzyme |
| | | | | | label |
| | | | | | dependent |
| | | | | | (usually |
| | | | | | APAP) |
| NMP22 | Bladder cancer | Blood, | Immunoassay | Home use | Enzyme |
| | | Urine | | | label |
| | | | | | dependent |
| | | | | | (usually |
| | | | | | APAP) |
| Estrogen | Fertility, ovulation | Urine | Immunoassay | Home use | Enzyme |
| | | | | | label |
| | | | | | dependent |
| | | | | | (usually |
| | | | | | APAP) |
| Warfarin | Anticoagulation | Blood | Immunoassay | Home use | Enzyme |
| | | | | | label |
| | | | | | dependent |
| | | | | | (usually |
| | | | | | APAP) |
| Chloride | Body electrolytes | Sweat | Direct | Home use | No |
| | | | | | substrate |
| | | | | | needed |
| Troponin | Myocardial damage | Blood | Immunoassay | Home use or | Enzyme |
| | | | | clinical setting | label |
| | | | | | dependent |
| | | | | | (usually |
| | | | | | APAP) |
| Glycogen | Fleart failure | Blood | Immunoassay | Home use or | Enzyme |
| phosphorylase | | | | clinical setting | label |
| isoenzyme BB | | | | | dependent |
| | | | | | (usually |
| | | | | | APAP) |

The analyte may be situated inside a cell (i.e. intracellular) or on the cell membrane (i.e. membrane bound). According to another embodiment, the analyte is a cell-secreted analyte. It will be appreciated that when the analyte to be detected is intracellular, the substrate is preferably membrane permeable. Furthermore, the substrate is preferably selected such that following catalysis, the product formed is also membrane permeable such that it may diffuse away from the cell and on to the detector electrode.

Contemplated cells, according to this aspect of the present invention include prokaryotic or eukaryotic cell which can be genetically modified (in a transient or stable manner) to express exogenous polynucleotides such as a reporter polypeptide.

According to a particular embodiment, the cell is a cancer cell, as further described herein above.

Examples of prokaryotic cells which can be used in accordance with the invention include but are not limited to bacterial cells, such as Pseudomonas, Bacillus, Bacteriodes, Vibrio, Yersinia, Clostridium, Mycobacterium, Mycoplasma, Coryynebacterium, Escherichia, Salmonella, Shigella, Rhodococcus, Methanococcus, Micrococcus, Arthrobacter, Listeria, Klebsiella, Aeromonas, Streptomyces and Xanthomonas.

Examples of eukaryotic cells which can be used in accordance with the invention include but are not limited to cell-lines, primary cultures or permanent cell cultures of fungal cells such as Aspergillus niger and Ustilago maydis [Regenfelder, E. et al. (1997) EMBO J. 16:1934-1942], yeast cells (see U.S. Pat. Nos. 5,691,188, 5,482,835 and Example 5 of the Examples section which follows), such as Saccharomyces, Pichia, Zygosaccharomyces, Trichoderma, Candida, and Hansenula, plant cells, insect cells, nematoda cells such as c. elegans, invertebrate cells, vertebrate cells and mammalian cells such as fibroblasts, epithelial cells, endothelial cells, lymphoid cells, neuronal cells and the like. Cells are commercially available from the American Type Culture Co. (Rockville, Md.).

According to one embodiment, the analyte is an enzyme that is capable of converting a substrate into a product which can be detected electrically (i.e. an electric signal).

According to one embodiment, the enzyme required to be detected is alkaline phosphatase (or enzyme secreted alkaline phosphatase, (SEAP)) and the substrate is, 4-aminophenyl phosphate (p-APP). Alkaline phosphatase converts p-APP to the electrochemical product, p-aminophenol (PAP).

P-APP is widely commercially available from such Companies as Sigma-Aldrich, Bio-world and many others.

Alkaline phosphatase is present in normal cells, but is reduced (or even absent) in cancerous cells. Therefore, analysis of the alkaline phosphatase activity level of cells, may be used as a marker for evaluating the efficiency of a particular drug for treating cancer and even for diagnostic purposes.

According to one embodiment, the enzyme is glucose oxidase immobilized to the electrode and the substrate is glucose-6-phosphate. According to another embodiment, the enzyme is chloramphenicol acetyl transferase (CAT) and the substrate chloramphhenicol. According to still another embodiment, the enzyme is b-glucuronidase and is substrate is any glycosaminoglycans or other glycoconjugates that after the removal of the b-glucorunic acid residue become electrochemically active.

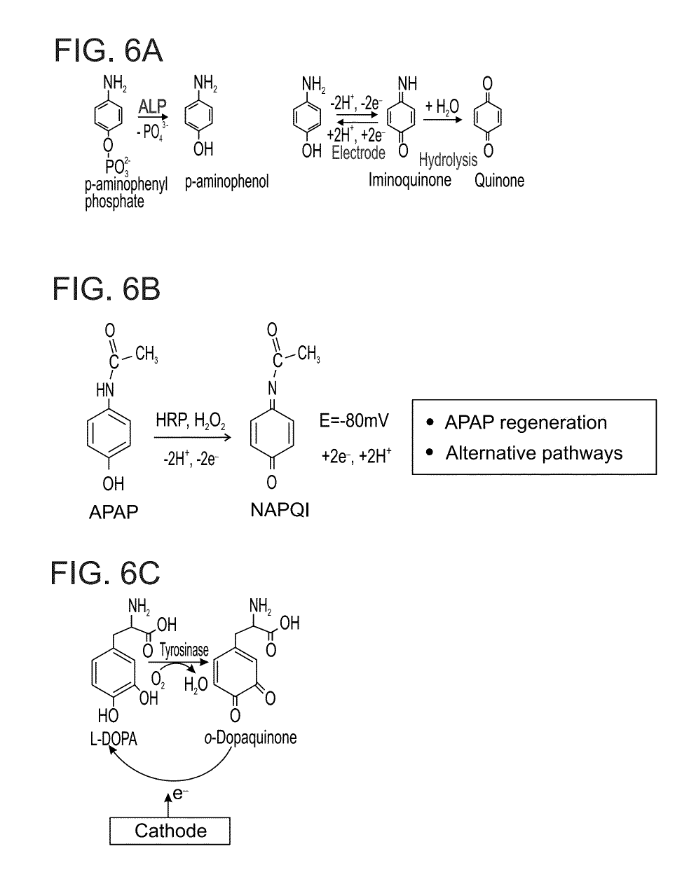

Other examples for such substrates include, without limitation, various derivatives of aminophenols (e.g. para-aminophenol, 1-naphthol, acetaminophenol etc.), L-DOPA etc. reaction mechanism for such substrates are shown in FIGS. 6A-C.

It will be appreciated that an analyte or a parameter can be detected using a reporter construct wherein the analyte or parameter correlates with expression of the reporter.

As used herein, the term “correlates” refers to the correlation between the measured signal and the parameter/analyte which is to be determined. Such correlation may be manifested either by a proportional increase in the signal in line with the level of said parameter/analyte, or a proportional decrease in the signal in line with said parameter/analyte. Alternatively, the correlation may be inversely proportional. For example an increase in analyte may be represented by a decrease in signal.

Thus, the present invention contemplates transfecting cells with a nucleic acid construct comprising an inducible cis acting regulatory element operatively linked to a reporter polypeptide which is capable of being electrically detected.

Transfection of the cells may be achieved by any known transfection techniques. Such techniques may involve the use of viral vectors such as, for example, the baculla virus system for the transfection of insect cells, the adenovirus system for transfection of human cells, lambda bacterial system for transfection of bacteria, etc. In addition, a variety of transfection techniques involving the use of plasmids may also be used for transfection of the host cells. A typical method of transfection of mammalian cells may be the calcium chloride technique ionophoretic transfection techniques, etc. (Sambrook, J., Fritsch, E. F., and Maniatis, T., Molecular cloning: a laboratory manual (second edition) Cold Spring Press, Cold Spring Harbor, N.Y. (1989)). As will be appreciated, the invention is not limited to a particular host cell or to the type of transfection method utilized.

As used herein a “cis acting regulatory element” refers to a naturally occurring or artificial polynucleotide sequence, which binds a trans acting regulator and regulates the transcription of a coding sequence located down-stream thereto. For example, a transcriptional regulatory element can be at least a part of a promoter sequence which is activated and/or repressed by a specific transcriptional regulator or it can be an enhancer which can be adjacent or distant to a promoter sequence and which functions in up regulating the transcription therefrom.

It will be appreciated that the cis-acting regulatory element of this aspect of the present invention may be stress regulated (e.g., stress-regulated promoter), which is essentially activated in response to cellular stress produced by exposure of the cell to, for example, chemicals, environmental pollutants, heavy metals, changes in temperature, changes in pH, as well as agents producing oxidative damage, DNA damage, anaerobiosis, and changes in nitrate availability or pathogenesis.

The promoter included in the nucleic acid construct in which the cell is being transfected may be selected from a wide variety of known promoters. According to one embodiment, the promoter is an inducible promoter that induces expression of the reporter polypeptide in the host cell upon occurrence of the parameter which is to be determined. For example, the promoter may be such which is inducible at a specific phase of the cell cycle, it may be inducible in the presence of a certain substance in the cell, e.g. a nutritional substance or a regulatory substance, external toxic chemical or pollutant, it may be a promoter inducible by external culture conditions, e.g. when the culture reaches a stationary growth stage, or by an external factor such as a toxic chemical, a pollutant, etc.

Examples of promoters which may be used in accordance with this aspect of the present invention include, but are not limited to, MipA, LacZ, GrpE, Fiu, MalPQ, oraA, nhoA, otsAB and yciD, KatG, nblA, glnA, phoA, micF, fabA, ars, cup1, cad, pbr, mer, umuDC, polB, sulA (sfiA), recN, recA, Cda, alkA, alkB, nrdA, and uvrA. Detailed description of such promoters is provided in WO2005/069738, which is hereby incorporated by reference in its entirety.

A cis acting regulatory element can also be a translational regulatory sequence element in which case such a sequence can bind a translational regulator, which up regulates translation.

The expressible DNA sequence may encode a catalytically active expression product. In accordance with one embodiment, such an expression product is an enzyme which catalyzes a reaction giving rise to a product which is permeable or which can be transported through the cell membrane and which can then undergo redox reaction at one of the electrodes of the electrochemical cell, as exemplified herein above.

According to another embodiment, the expressible DNA sequence is a polypeptide which can be recognized by an antibody. The antibody may be coated on the electrode, using methods known in the art.

In accordance with one embodiment of the invention, the reporter gene may comprise several genes of which one encodes the substrate or a peptide capable of producing the substrate and another gene encodes the enzyme capable of catalyzing a reaction on the produced substrate. Several reporter genes may be expressed from the sane promoter, one of the reporter genes encoding the substrate. In this manner, it is not necessary to add a substrate to the cells and the final signal is actually the result of a sequence of proteins in the complex.

In accordance with one embodiment the entire culture consists of the host cells which also produce a substance of interest as well as being capable of expressing said expressible product thus allowing to monitor said parameter. In accordance with another embodiment, the culture comprises a certain proportion of the host cells which allow to monitor said parameters. In such an embodiment, it is necessary to continuously ascertain that a fixed proportion between the host cells and the culture cells are maintained. Furthermore, in accordance with this embodiment, it is possible, at times, to include in the culture a number of different host cells, each expression a different expressible product to allow to differentiate between the different parameters.

The determination of the parameters may, by one embodiment (the “on-line” embodiment), be performed by forming the electrochemical cell within the fermentation vessel. This will require to include in such a vessel typically three electrodes, a reference electrode, a working electrode and a counter electrode.

Alternatively, rather than performing the measurement within the culture, by an additional embodiment the “semi on-line” embodiment) it is possible also to continuously withdraw samples and place such samples within the electrochemical cells. In case where the culture contains different host cells, in each electrochemical cell, it is possible to add a different substrate to allow to differentiate between the signals from different types of host cells.

The system of the present embodiments optionally and preferably utilizes at least one of the following detection methods:

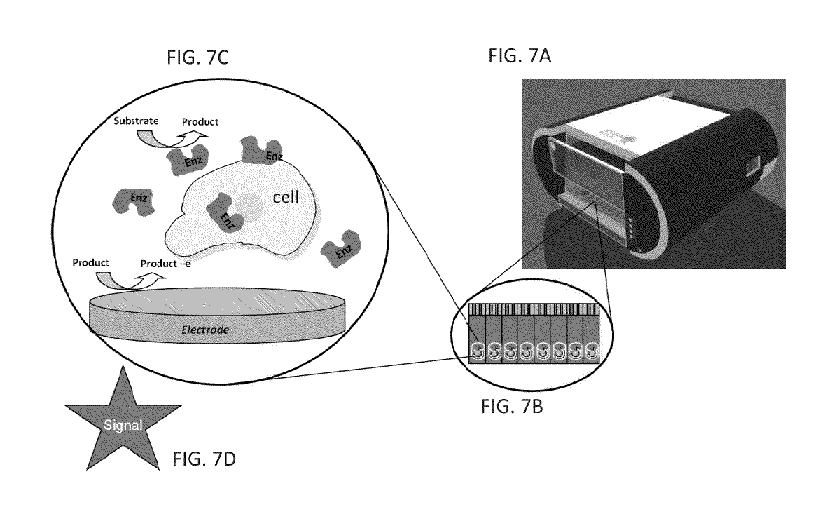

1. Direct electrochemical detection of enzymatic activity. This method can be implemented when the biomarker to be detected is an enzyme and its activity may be quantitated by a tailored substrate which is added to the system. Upon catalysis of the substrate by the enzyme biomarker, an electro-active product is obtained. This product undergoes a redox reaction on a working electrode at a specific applied voltage thus yielding measurable current. A general scheme illustrating the above detection method is shown in FIG. 7.

Alternatively, this method may be carried out by using an enzyme substrate, instead of a chemical reagent. In the case where the detected biomarker is a protein (including glycoprotein or proteoglycan), a certain enzyme, which is specific to this biomarker and is able to convert it into an electroactive product (or generate electroactive byproducts upon catalysis) is added to the system.

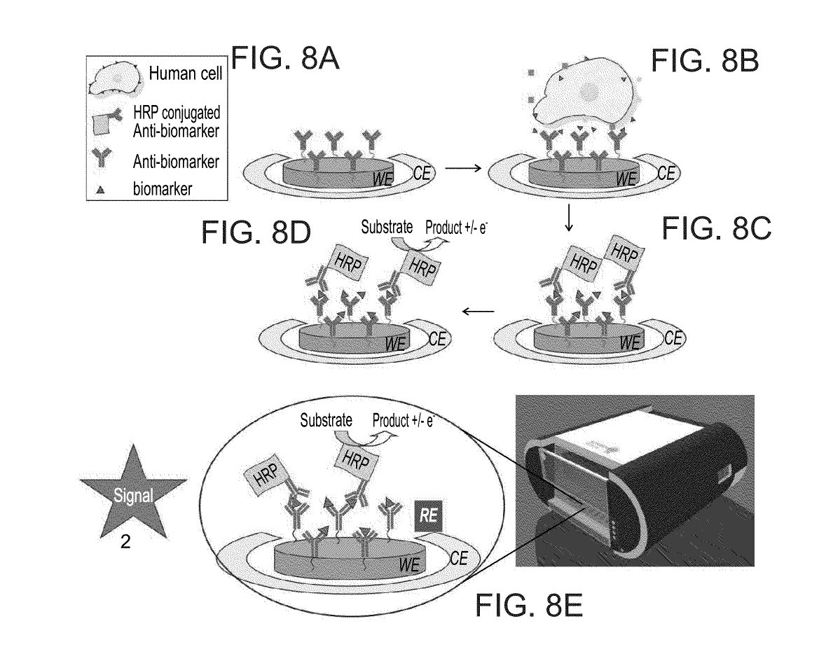

2. Electrochemical immunoassay (ECI). This configuration may be performed in various manners. In one example the biochip contains an antibody specific to a certain biomarker. Once the biospecimen is introduced the secreted biomarker is captured by the antibody. Subsequently, a secondary antibody, which is an immunoconjugate (antibody labeled by an enzyme tag) also binds the same biomarker. Once an electrochemical substrate is introduced, the enzyme catalyzes the conversion of the substrate into an electroactive product which is than oxidized or reduced on the working electrode thus generating a measurable current detected by the potentiostat. A general scheme illustrating this detection method is shown in FIG. 8.

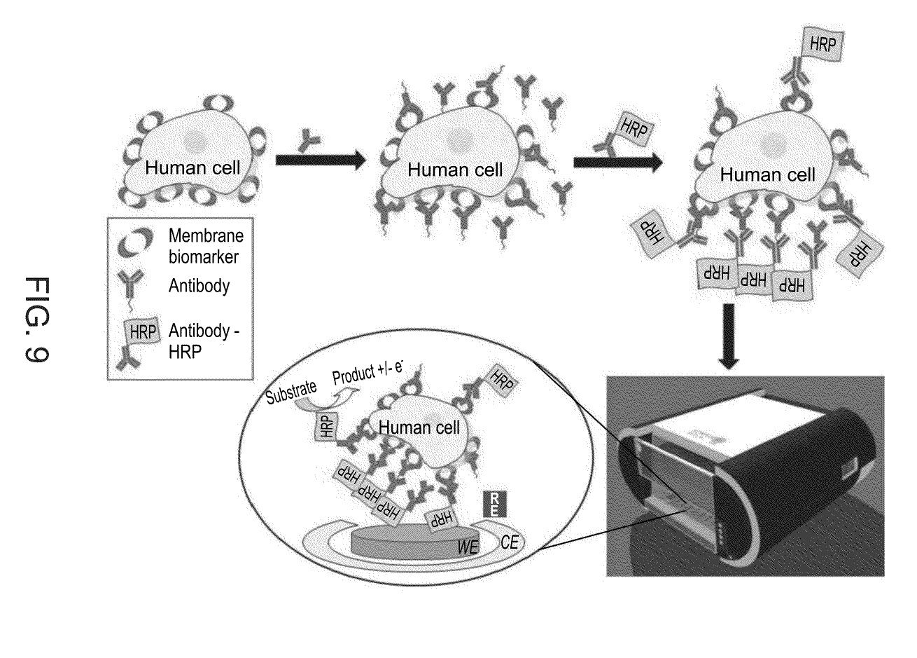

In another application of the electrochemical immunoassay, a modification of the process is implemented. In this example, aimed for the detection of cell membrane bound biomarkers, the biospecimen used is a solid tissue or cell sample contained in blood or other body fluids. The expression levels of the membrane bound biomarkers in the cells contained in the sample are quantitated by an immunoassay. A general description of the method is presented in FIG. 9.

The system of the present embodiments can also comprise one or more of the following features:

a. Measurement Buffer, such as, but not limited to, a routinely employed phosphate buffered saline (PBS).

b. Electrochemical substrate, e.g., a chemical reagent which is specific to an enzyme and is able to undergo biochemical catalysis by a specific enzyme. Additionally, the electrochemical substrate can yield, following enzymatic biocatalysis, an electro-active product. Such an electro-active product can, for example, undergo electrochemical oxidation or reduction at a predefined applied voltage.

c. An enzyme substrate. This embodiment is particularly useful when the biomarker to be detected is a certain protein biomolecule, in which case the enzyme substrate of the biochip may be an enzyme specific to the biomolecule and is able to convert it into an electro-active product.

d. In some embodiments, antibodies specific to the detected biomarker may be used. The antibodies may be immobilized to the electrodes via chemical modifications.

e. In some embodiments, antibodies which are labeled by a redox enzyme (e.g. HRP, ALP, Laccase, Catalase, GOX etc.) may be used. These antibodies may be immobilized to the electrodes.

It is expected that during the life of this patent many relevant substrates will be developed and the scope of the term substrate is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

For feasibility, demonstration tumors were induced in athymic nude mice by injecting the human melanoma cancer cell line, MEL526. The cancer cell line was injected separately to athymic nude mice subcutaneously. Tumors were allowed to develop and mice were sacrificed following 4 weeks. Subcutaneous tumors were collected. As control, normal skin tissues were simultaneously collected from the skin of the same mice. Following removal, biopsy samples were washed with PBS and dissected to samples ˜4 mm Chronoamperometry was performed with a PalmSens portable potentiostat (Palm Instruments BV, the Netherlands) equipped with an eight-channel multiplexer allowing for the simultaneous measurement of eight electrochemical cells. An in-house apparatus providing electrical contacts of the screen print electrodes combined with suction-expulsion-based efficient stirring was used. The electrochemical chamber was constructed as a 300 μl chamber equipped with electrochemical cells with planar carbon working, carbon counter, and Ag/AgCl reference electrodes. During measurements continuous mixing was affected. Each biopsy slice was suspended in 270 μl PBS in the electrochemical chamber. All electrodes were connected via the eight channel multiplexer, continuously operating under mixing. A potential of −300 mV vs. Ag/AgCl reference electrode was applied. Following a short equilibration time, allowing for the stabilization of the system and determination of the background signal emerging from background electrochemical and biochemical reactions, the substrate L-DOPA was added (3 μl) to make a final concentration of 0.5 mM. The enzyme tyrosinase catalyzes the oxidation of L-DOPA to Dopaquinone which is than reduced at the working electrode at low negative potentials.

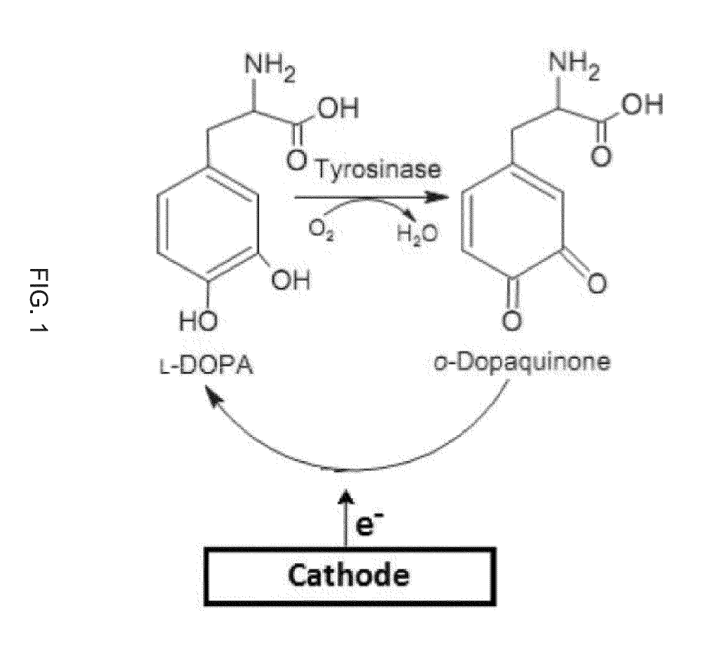

Reduction of L-DOPA yields a measurable cathodic current which is directly correlated to the tyrosinase activity within the measured tissue sample. The enzymatic and subsequent electrochemical reactions are shown in FIG. 1.

The detection system comprised the electrochemical biochip, the platform including the stirring apparatus, the potentiostat with multiplexer and computer. The system is presented in FIG. 2.

Results

Tissue samples were dissected to small ˜4 mm slices. The small sample size was sufficient to induce an electrochemical response, originating from the enzymatic activity of tyrosinase in the tumor derived tissue samples. Thus, tissue samples derived from xenograft tumors could be easily distinguished from the samples removed from a normal skin tissue, used as a negative control, as shown in FIG. 4. The obtained current signals were distinctively higher indicating the activity of tyrosinase in the malignant tissue while no current signals were obtained for the healthy derived tissue samples. Multiple measurements yielded reproducible current signals thus supporting the feasibility of the biosensor and of the working hypothesis.

Upon measurement of multiple samples it was clear that normal skin tissue didn't express any tyrosinase enzymatic activity while malignant tissue express variable levels of activity. The average slopes of 40 samples is presented in FIG. 5. Distinctively higher curves were obtained for malignant samples.

### CONCLUSION

This research demonstrated the feasibility of a straightforward, easy to use, electrochemical biosensor platform able to detect the activity of tyrosinase melanoma biomarker directly from tissue samples. The rapid measurement time (5 min) and the elimination of the need for sample pre-treatment and handling, may offer unique advantages to the system. Consequently, this system may be used by dermatologists and dermapathologists for the onsite diagnosis of melanoma. The same method and biosensor may also be used during SLN (sentinel lymph node biopsy) procedure and MOHS procedure as an efficient tool for intra-procedural or intraoperative biomarker detection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of determining tyrosinase activity of a cell comprising:
(a) contacting the cell with a phenol under conditions wherein tyrosinase of the cell catalyzes a reaction with said phenol, so as to generate a product which produces an electrical signal; and
(b) measuring a level of said electrical signal, thereby determining tyrosinase activity of the cell.

2. The method of claim 1, wherein said cell is a skin cell.

3. The method of claim 1, wherein the cell is a mammalian cell.

4. The method of claim 3, wherein said phenol comprises tyrosine or 3,4-dihydroxyphenylalanine (DOPA).

5. The method of claim 4, wherein said DOPA is L-DOPA.

6. A method of diagnosing a subject with skin cancer comprising:
(a) contacting at least one skin cell which is suspicious of a cancerous phenotype of the subject with L-DOPA or tyrosine under conditions wherein tyrosinase of said at least one cell catalyzes a reaction with said L-DOPA or tyrosine, so as to generate a product which produces an electrical signal; and
(b) measuring a level of said electrical signal, wherein an increase in a strength of said electrical signal above a predetermined threshold is indicative of skin cancer, thereby diagnosing the subject with skin cancer.

7. A method of individually optimizing a treatment for skin cancer, the method comprising:
(a) contacting at least one skin cancer cell of a subject with at least one anti cancer agent;
(b) contacting said at least one skin cancer cell with L-DOPA or tyrosine, under conditions wherein tyrosinase of said at least one cell catalyzes a reaction with said DOPA or tyrosine, so as to generate a product which produces an electrical signal; and
(c) measuring a level of said electrical signal produced by the cell, wherein a decrease in said level is indicative of an efficient anti cancer agent for the treatment of the skin cancer of said subject, thereby individually optimizing a treatment for cancer.

8. (canceled)

9. The method of claim 2, wherein said skin cell is comprised in a skin tissue slice.

10. The method of claim 6, wherein said at least one skin cell is comprised in a skin tissue slice.

11-12. (canceled)

13. The method of claim 9, wherein said skin tissue slice is frozen prior to said contacting.

14. The method of claim 9, wherein said skin tissue slice is fixed by chemical fixatives prior to said contacting.

15. The method of claim 9, wherein said skin tissue slice is not pretreated prior to said contacting.

16-17. (canceled)

18. The method of claim 1, wherein said contacting is effected in vitro.

19. The method of claim 1, wherein said contacting is effected ex vivo.

20. The method of claim 10, wherein said skin tissue slice comprises no more than one million cells.

21. The method of claim 10, wherein said skin tissue slice comprises no less than 10 cells.

22. The method of claim 6, wherein said measuring is effected using an electrochemical cell configured for sensing said produced signal.

23. The method of claim 22, wherein said measuring comprises:
(i) establishing communication between said electrochemical cell and a measuring device configured for receiving and measuring an electrical signal generated by said electrochemical cell; and
(ii) establishing communication between said measuring device and a hand-held electronic device supplemented by software for receiving, analyzing and presenting data pertaining to said measurement.

24. The method of claim 23, wherein said hand-held electronic device is selected from the group consisting of a cellular telephone with data processing functionality, a personal digital assistant (PDA) with data processing functionality, a portable email device with data processing functionality, a portable media player with data processing functionality, a portable gaming device with data processing functionality, a tablet, and a touch screen display device with data processing functionality.

25. The method of claim 23, wherein said electrochemical cell and said measuring device are confined in the same physical encapsulation.

26. The method of claim 23, wherein said electrochemical cell and said measuring device are separated from each other and being in electrical communication thereamongst.

27-52. (canceled)

53. The method of claim 6, wherein said skin cancer is a melanoma.
